SEQUENCE LISTING (15 sequences)

SEQ ID NO: 1 - DNA, 25 bp, Artificial Sequence
  Oligonucleotide
ccacggagactggaacagatgtcgg

SEQ ID NO: 2 - DNA, 23 bp, Artificial Sequence
  Oligonucleotide
ggatcccagtgctctagactgtc

SEQ ID NO: 3 - DNA, 46 bp, Artificial Sequence
  Oligonucleotide
gctagggataacagggtaatgcgcggtagctgagacatgtcgcgtg

SEQ ID NO: 4 - DNA, 44 bp, Artificial Sequence
  Oligonucleotide
gcattaccctgttatccctagcgcgttcgtcctctcatgattcc

SEQ ID NO: 5 - DNA, 24 bp, Artificial Sequence
  Oligonucleotide
ccaaacatagtcatttgcggatcc

SEQ ID NO: 6 - DNA, 22 bp, Artificial Sequence
  Oligonucleotide
ccaagggaccgtctggccaaac

SEQ ID NO: 7 - DNA, 25 bp, Artificial Sequence
  Oligonucleotide
cggacacctcttttatgctgcgggc

SEQ ID NO: 8 - DNA, 23 bp, Artificial Sequence
  Oligonucleotide
ggcggatcctggtgcatttttgc

SEQ ID NO: 9 - DNA, 45 bp, Artificial Sequence
  Oligonucleotide
gctagggataacagggtaatgcgcaaactcatctgggggagatcc

SEQ ID NO: 10 - DNA, 45 bp, Artificial Sequence
  Oligonucleotide
gcattaccctgttatccctagcgagcttatcagtcacggtccacg

SEQ ID NO: 11 - DNA, 21 bp, Artificial Sequence
  Oligonucleotide
ccatagaggtgtccccaaacg

SEQ ID NO: 12 - DNA, 23 bp, Artificial Sequence
  Oligonucleotide
cccccaagcataccgaccgtcgc

SEQ ID NO: 13 - DNA, 23 bp, Artificial Sequence
  Oligonucleotide
cttatataccaaagggatgggtc

SEQ ID NO: 14 - DNA, 23 bp, Artificial Sequence
  Oligonucleotide
gtagatagttgaggtagaagttg

SEQ ID NO: 15 - DNA, 21 bp, Artificial Sequence
  Oligonucleotide
gggtataaaagaccaccgtcc

(19) United States
(12) Patent Application Publication
(10) Pub. No.: US 2012/0226059 A1
(43) Pub. Date: Sep. 6, 2012

(54) Method for the Production of Very Long Chain Fatty Acids (VLCFA) by Fermentation with a Recombinant Yarrowia SP

(76) Inventors: Jean-Denis Faure, Gif-sur-Yvette (FR); Jean-Marc Nicaud, Trappes (FR); Brice Bourdenx, Fontenay le Fleury (FR); Ramdane Haddouche, Thiverval-Grignon (FR)

(21) Appl. No.: 13/510,577

(22) PCT Filed: Nov. 30, 2010
(86) PCT No.: PCT/EP2010/068541

§ 371 Date: May 17, 2012

(30) Foreign Application Priority Data
Nov. 30, 2009 (EP) 09306158.8

(51) Int. Cl. C12N 1/15; C07C 53/126; C12P 7/64

(52) U.S. Cl. 554/1; 435/254.11; 435/134

(57) ABSTRACT

The present invention concerns a method for the production of Very Long Chain Fatty Acids (VLCFA) by fermentation, comprising culturing a recombinant strain of a Yarrowia sp. comprising a heterologous gene coding for a hydroxyacyl-CoA dehydratase, under control of regulatory elements allowing expression of the said heterologous gene in the said Yarrowia sp. The invention also concerns the recombinant Yarrowia sp.

### INTRODUCTION

The present invention concerns a method for the production of Very Long Chain Fatty Acids (VLCFA) by fermentation, comprising culturing a recombinant strain of a Yarrowia sp. comprising a heterologous gene coding for a hydroxyacyl-CoA dehydratase, under control of regulatory elements allowing expression of the said heterologous gene in the said Yarrowia sp.

The invention also concerns the recombinant Yarrowia sp.

### BACKGROUND OF THE INVENTION

Living organisms synthesize a vast array of different fatty acids which are incorporated into complex lipids. These complex lipids represent both major structural component membranes, and are a major storage product in both plants and animals.

Very-long-chain fatty acids (VLCFAs) are components of eukaryotic cells and are composed of 20 or more carbons in length (i.e. >C18). VLCFAs are involved in many different physiological functions in different organisms. They are abundant constituents of some tissues like the brain (myelin) or plant seed (storage triacylglycerols, TAGs). VLCFAs are components of the lipid barrier of the skin and the plant cuticular waxes. The long acyl chain of certain VLCFAs is necessary for the high membrane curvature, found for instance in the nuclear pore. VLCFAs are also involved in the secretory pathway for protein trafficking and for the synthesis of GPI lipid anchor. Finally, VLCFAs are components of sphingolipids that are both membrane constituents and signalling molecules.

VLCFA are fatty acids with an acyl chain longer than C18. Polyunsaturated, they are considered as important nutritional components of the human diet mainly as Eicosapentaenoic acid (EPA) or Docosahexaenoic acid (DHA). The patent application WO 2005/118814 discloses a way to improve the production of polyunsaturated fatty acids in Saccharomyces cerevisiae. Unsaturated, VLCFA are also of industrial interest since they act as detergent or lubricants.

VLCFA are synthesized by the sequential addition of two carbons through four successive enzymatic reactions gathered in the endoplasmic reticulum within a protein complex named elongase complex, a membrane-bound enzymatic complex containing four distinct enzymes (KCS, KCR, HCD and ECR). The first step of fatty elongation is the condensation of a long chain acyl-CoA with a malonyl-CoA by the 3-keto-acyl-CoA synthase (KCS or condensing enzymes). The resulting 3-keto-acyl-CoA is then reduced by a 3-keto-acyl-CoA reductase (KCR) generating a 3-hydroxy-acyl-CoA. The third step is the dehydration of the 3-hydroxy-acyl-CoA by a 3-hydoxy-acyl-CoA dehydratase (HCD) to an trans-2,3-enoyl-CoA which is finally reduced by the trans 2,3-enoyl-CoA reductase (ECR) to yield a two carbon elongated acyl-CoA. The last three enzymes are referred as core enzymes since they are not involved in acyl-CoA specificity. Once acyl-CoA have been elongated from the elongase complex, they can be incorporated into different lipid classes, like phospholipids, triacylglycerols, sphingolipids and specific lipids like plant epicuticular waxes.

Yarrowia lipolytica is considered as an oleaginous yeast because this yeast can accumulate more than 50% of its dry weight as lipids, but also is able to use efficiently lipids as carbon source (Beopoulos & al. 2009). The complete sequencing of its genome as well as the development of molecular genetic tools for this yeast has made this organism not only a model for studying the mechanism of lipid accumulation, but also a cell factory for oleochemical biotechnology. Recently, it was shown that the combined deletions of the glucose 3-phosphate dehydrogenase GUT2 and the POX1-6 genes involved in the β-oxidation led to very high accumulation of lipids, mainly free fatty acids (Beopoulos & al. 2008, FR0854786; 11 Jul. 2008). This obese strain accumulated twice and three times more fatty acids than wild type when grown respectively on glucose or oleic acid. Interestingly, these lipids were accumulated in a single large lipid body. Yarrowia lipolytica accumulates mainly the long chain fatty acids c18:2, c18:1 (n-9), c16:1 (n-7) and c16:0. However little information is available on very long chain fatty acids (VLCFA) in Y. lipolytica.

It was now found that expressing a heterologous gene coding for a hydroxyacyl-CoA dehydratase in a Yarrowia sp. and particularly Yarrowia lipolytica had a direct impact on the strain's production of fatty acids and VLCFA, in terms of quality and/or quantity.

### BRIEF DISCLOSURE OF THE INVENTION

The present invention concerns a recombinant strain of a Yarrowia sp., comprising a heterologous gene coding for a hydroxyacyl-CoA dehydratase, under control of regulatory elements allowing expression of the said heterologous gene in the Yarrowia sp.

The gene coding for the hydroxyacyl-CoA dehydratase is particularly selected among the group consisting of genes of plant sp. coding for a hydroxyacyl-CoA dehydratase, functional homologues and fragments thereof. The gene of plant sp. is advantageously selected among the genes coding for an hydroxyacyl-CoA dehydratase from Arabidopsis thaliana, Vitis vinifera, Oryza sativa, Brassica rapa, Hyacinthus orientalis, Ostreacoccus lucimarinus, Chlamydomonas reinhardtii, Brassica napus, Raphanus sativus, and Brassica oleracea and more particularly the gene PAS2 from Arabidopsis thaliana.

The invention also concerns a method for the production of Very Long Chain Fatty Acids (VLCFA) by fermentation, comprising culturing a recombinant strain of the invention in an appropriate culture medium and recovering the VCLFA from the strains and/or the medium.

The fatty acids produced by the said method are also parts of the invention.

### DETAILED DISCLOSURE OF THE INVENTION

The present invention concerns a recombinant strain of a Yarrowia sp., comprising a heterologous gene coding for a hydroxyacyl-CoA dehydratase, under control of regulatory elements allowing expression of the said heterologous gene in the Yarrowia sp.

### Recombinant Strain

According to the invention, the strain is recombinant when it has been genetically modified by means of cellular biology such as gene replacement or plasmid introduction. It may be obtained by directed mutagenesis to introduce a new gene or mutations or new regulatory elements in a gene or to delete an endogenous gene. A recombinant microorganism is not the sole result of random mutagenesis.

When the new gene is introduced in the strain, it may be introduced with an expression plasmid, or integrated in the genome of the strain.

When integrated in the genome of the strain, the gene may be integrated randomly or on a specific site by known methods of gene replacement, like homologous recombination techniques.

The heterologous gene when introduced can comprise the coding sequence under control of the regulatory elements allowing expression of the said heterologous gene in the Yarrowia sp. Alternatively, it can comprise the coding sequence which is introduced in the genome of the microorganism under control of existing endogenous regulatory elements, replacing the corresponding endogenous coding sequence which is deleted.

Methods for the modification of a Yarrowia sp. particularly to introduce new genes or delete genes are known in the art, including Barth and Gaillardin (1996) and Fickers et al. (2003).

### Heterologous Gene Coding for a hydroxyacyl-CoA dehydratase

The term ‘hydroxyacyl-coA dehydratase’ (HCD) designates an enzyme catalyzing a reaction of dehydration of the 3-hydroxy-acyl-CoA into trans-2,3-enoyl-CoA. It belongs to the family of hydro-lyases. This enzyme is part of the elongase complex and participates only to the synthesis of VLCFA in plants, and not to their degradation.

The gene coding for the hydroxyacyl-CoA dehydratase is heterologous. According to the invention, a gene is heterologous when it is not found as such in the native strain. It can be a native coding sequence under control of heterologous regulatory elements or a heterologous coding sequence under control of native regulatory elements. It can also be a gene with native components, found in the strain to be modified, but on a plasmid or in a locus in the genome where the same gene is not found in the unmodified strain.

The heterologous gene coding for a hydroxyacyl-CoA dehydratase is particularly selected among the group consisting of genes comprising a coding sequence from a gene of plant sp. coding for a hydroxyacyl-CoA dehydratase, functional homologues and fragments thereof.

Genes of plant sp. coding for a hydroxyacyl-CoA dehydratase are known in the art and includes particularly selected among genes from Vitis vinifera (encoding CAN64341.1 hypothetical protein), Oryza sativa (CAD39891.2, EAY72548.1 hypothetical protein OsI_{—}000395, EAZ30025.1 hypothetical protein OsJ_{—}013508 and BAD61107.1 tyrosine phosphatase-like), Brassica rapa (AAZ66946.1), Hyacinthus orientalis (AAT08740.1 protein tyrosine phosphatase), Ostreacoccus lucimarinus (XP_{—}001420997.1 predicted protein and XP_{—}001422898.1 predicted protein), Chlamydomonas reinhardtii (EDP01055.1 predicted protein), and also from Brassica napus, Raphanus sativus, Brassica oleracea.

In a preferred embodiment of the invention, the heterologous gene is the gene PAS2 from Arabidopsis thaliana (Bach et al., 2008), registered in UniGene databank under number NP_{—}196610.2, also known as F12B17.170; F12B17_{—}170; PASTICCINO 2; PEP; and PEPINO.

In another specific embodiment of the invention, the heterologous gene is the PHSI gene from Saccharomyces cerevisiae (Denic et al., 2007), registered in gene databanks under number NP_{—}012438.1, functional homologues and fragments thereof.

When the coding sequence of the heterologous gene is from another origin, it can be indeed recoded with preferred codon usages known for Yarrowia sp. The skilled person knows the preferred codon used in Yarrowia sp and how to prepare such a recoded coding sequence.

According to the invention, “functional homologues” are genes sharing homology with the heterologous gene coding for the hydroxyacyl-CoA dehydratase, or a gene encoding for a protein sharing homology with the protein encoded by the heterologous gene coding for a hydroxyacyl-CoA dehydratase.

A protein sharing homology with the protein encoded by the gene coding for a hydroxyacyl-CoA dehydratase may be obtained from plants or may be a variant or a functional fragment of a natural protein originated from plants.

The term “variant or functional fragment of a natural protein” means that the amino-acid sequence of the polypeptide may not be strictly limited to the sequence observed in nature, but may contain additional amino-acids. The term “a fragment” means that the sequence of the polypeptide may include less amino-acid than the original sequence but still enough amino-acids to confer hydroxyacyl CoA dehydratase activity. It is well known in the art that a polypeptide can be modified by substitution, insertion, deletion and/or addition of one or more amino-acids while retaining its enzymatic activity. For example, substitution of one amino-acid at a given position by a chemically equivalent amino-acid that does not affect the functional properties of a protein are common. For the purpose of the present invention, substitutions are defined as exchanges within one of the following groups:
Small aliphatic, non-polar or slightly polar residues: Ala, Ser, Thr, Pro, Gly
Polar, negatively charged residues and their amides: Asp, Asn, Glu, Gln
Polar, positively charged residues: His, Arg, Lys
Large aliphatic, non-polar residues: Met, Leu, Ile, Val, Cys
Large aromatic residues: Phe, Tyr, Trp.

Thus, changes that result in the substitution of one negatively charged residue for another (such as glutamic acid for aspartic acid) or one positively charged residue for another (such as lysine for arginine) can be expected to produce a functionally equivalent product.

The positions where the amino-acids are modified and the number of amino-acids subject to modification in the amino-acid sequence are not particularly limited. The man skilled in the art is able to recognize the modifications that can be introduced without affecting the activity of the protein. For example, modifications in the N- or C-terminal portion of a protein may be expected not to alter the activity of a protein under certain circumstances.

The term “variant” refers to polypeptides submitted to modifications such as defined above while still retaining the original enzymatic activity.

According to the invention, the polypeptide having an hydroacyl-CoA dehydratase enzymatic activity may comprise a sequence having at least 30% of homology with the sequence of PAS2, preferentially at least 50% of homology, and more preferentially at least 70% of homology.

Methods for the determination of the percentage of homology between two protein sequences are known from the man skilled in the art. For example, it can be made after alignment of the sequences by using the software CLUSTALW available on the website http://www.ebi.ac.uk/clustalw/ with the default parameters indicated on the website. From the alignment, calculation of the percentage of identity can be made easily by recording the number of identical residues at the same position compared to the total number of residues. Alternatively, automatic calculation can be made by using for example the BLAST programs available on the website http://www.ncbi.nlm.nih.gov/BLAST/ with the default parameters indicated on the website.

### Regulatory Elements Allowing Expression of the Heterologous Gene in the Yarrowia sp.

Such regulatory elements are well known in the art and include the POX2 promoter from acyl-CoA oxidase 2, the ICL promoter from Isocitrate dehydrogenase, the Promoter Hp4d, the Promoter GPD and GPM, the Promoter FBP and the Promoter XPR2. Said promoters are known in the art and disclosed, inter alia in Juretzek & al. (2000), Madzak & al. (2004). Madzak & al. (2000), U.S. Pat. No. 7,259,255, U.S. Pat. No. 7,202,356 and Blanchin-Roland et al (1994).

### Yarrowia sp.

According to the invention, any strain of a Yarrowia sp. may be transformed and used in the method of the invention. Preferably, the strain of Yarrowia sp. belongs to the genus Yarrowia lipolytica.

Strains of the genus Yarrowia lipolytica are well known in the art, as well as method for transforming such strains. Constructs comprising a coding region of interest may be introduced into a host cell by any standard technique. These techniques include transformation (e.g., lithium acetate transformation [Methods in Enzymology, 194:186-187 (1991)]), protoplast fusion, biolistic impact, electroporation, microinjection, or any other method that introduces the gene of interest into the host cell. More specific teachings applicable for oleaginous yeast (i.e., Yarrowia lipolytica) include U.S. Pat. Nos. 4,880,741 and 5,071,764.

Strains modified for an improved production of fatty acids have also been disclosed, like strains with very high accumulation of lipids, mainly free fatty acids (FR Patent Application No. 08/54786; 11 Jul. 2008), incorporated herein by reference. Such strains may be further modified according to the invention with a heterologous gene coding for a hydroxyacyl-CoA dehydratase.

The recombinant strain of the invention can also comprise deletion of at least one gene involved in the β-oxidation of fatty acids, particularly the deletion of one of the gene POX1 to POX6 coding for an acyl CoA oxidase, particularly the deletion of the six genes POX1-6 coding for the six acyl CoA oxidases And/or one gene involved in the patway of fatty acid and TAG synthesis, particularly the deletion of the gene coding for a glycerol 3-phosphate dehydrogenase.

### Culture of the Recombinant Strain

The fatty acids and particularly the VLCFA are produced when culturing the recombinant strain by fermentation in an appropriate culture medium.

Culture by fermentation means that the microorganism are developed on a culture medium and produce the VLCFA during this culture step, by transforming the source of carbon of the culture medium. The VLCFA is accumulated with the biomass, in the cells and/or in the medium.

Fermentation is distinct from bioconversion where the culture is used to produce enzymes, further used in a enzymatic conversion process.

### Appropriate Culture Medium

Culture mediums for Yarrowia sp. are well known in the art, including Barth and Gaillardin (1996), Nicaud et al. (2002) and Mauersberger and Nicaud (2002).

Define media for fermentation are particularly disclosed in Leblond & al. (2009) and KR 2009 0029808.

Sucrose media are particularly disclosed in Nicaud & al. (1989).

Appropriate culture mediums are those mediums where the Yarrowia sp. can grow and contains all the nutrients allowing growth of the strain and production of VLCFA, particularly a source of carbon.

The source of carbon may be any source of carbon, such as sucrose or other carbohydrates.

### Recovering the VCLFA from the Strains and/or the Medium

The VCLFA are accumulated with the biomass, in the strains and/or in the culture medium. Recovery of the VCFLA comprises generally steps of cells lysis, filtration and recovery from the medium. The person skilled in the art of fatty acids bioproduction knows how to adapt the usual methods for recovering a fatty acid from the biomass to the method of the invention.

The VCFLA produced with the method of the invention may be used as such, in mixtures of fatty acids produced by the strain of the invention. They can also be further purified and isolated.

### FIGURES

FIG. 1 represents the synthetic PAS2 optimized for Yarrowia lipolytica expression. (A) Sequence of PAS2^{Y1} gene and protein. (B) Alignment of Arabidopsis PAS2^{At} with PAS2^{Y1}.

FIG. 2 represents PAS2 expression in Yarrowia with a schematic view of the different strains used or created.

FIG. 3 represents the effect of PAS2 expression in Yarrowia. (A) Staining of lipid bodies with Red Nile in JM1367 and JM1781 strains. (B) Number of lipid bodies per cell in Po1d and JM1777. Polynomial regression was applied for comparing the distributions. (C) Number of lipid bodies per cell in JM1367 and JM1381. Polynomial regression was applied for comparing the distributions.

FIG. 4 shows the ratio of produced LCFA/VLCFA with PAS2 expression in Yarrowia.

FIG. 5 shows modified LCFA and VLCFA contents with PAS2 expression in Yarrowia. (A) LCFA content. (B) VLCFA content.

FIG. 6 shows the modified VLCFA profile with PAS2 expression in Yarrowia.

### EXAMPLES

### Material and Methods

The synthetic PAS2 gene (PAS2^{Y1}) was synthesised according to Yarrowia lipolytica codon usage giving rise to plasmid JME1107. PAS2^{Y1} was cloned into plasmid JMP62-POX2-URA3ex (JME803) and into JMP62-TEF-URA3ex (JME1012) as follow: Plasmid JME1107 was digested by BamHI-AvrII and the corresponding fragment carrying PAS2 gene was cloned at the corresponding site of plasmid JME1012 and JME1107, giving rise to plasmid JME1108 (POX2-PAS2) and JME1110 (TEF-PAS2) respectively. Plasmids were digested by NotI and the fragment carrying the expression cassette were used for transformation of Yarrowia lipolytica by the lithium acetate method (described in the revue of G. Barth and Gaillardin: (Yarrowia lipolytica, in: Nonconventional Yeasts in Biotechnology A Handbook (Wolf, K., Ed.), Vol. 1, 1996, pp. 313-388. Springer-Verlag). Transformants were selected onto YNBcasa. Typically, about 5×10³ transformants were obtained per μg of fragments. Four to height transformants were analysed by PCR with primer pairs 61start/61stop and TEFstart/61stop for clones containing the POX2-PAS2 and TEF-PAS2, respectively. The PCR products were further digested by AvaI unique restriction site in the PAS2 gene.

### Stable Expression of PAS2 in Yarrowia lipolytica

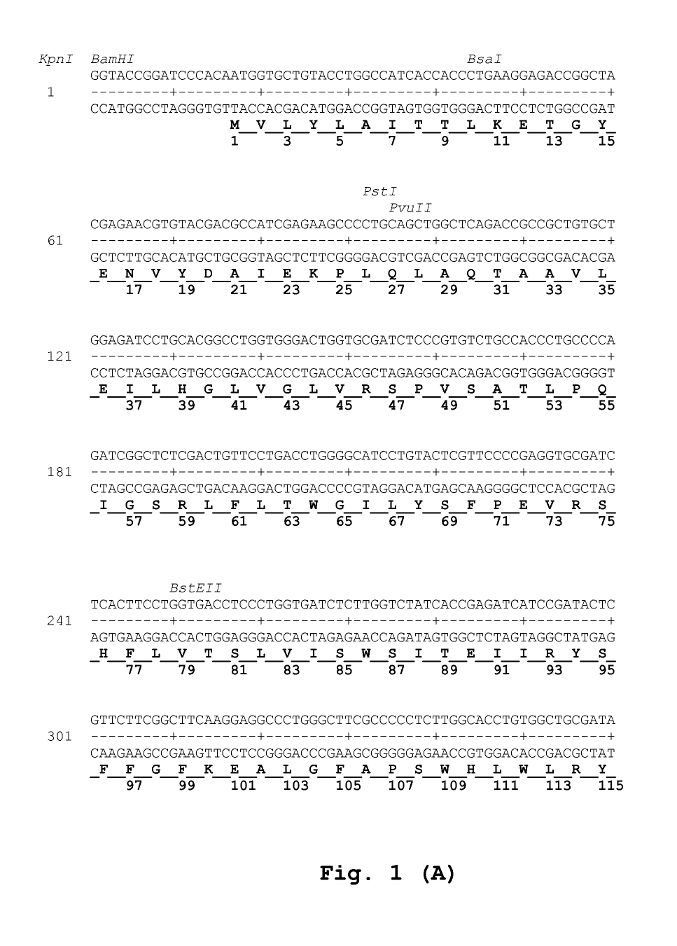

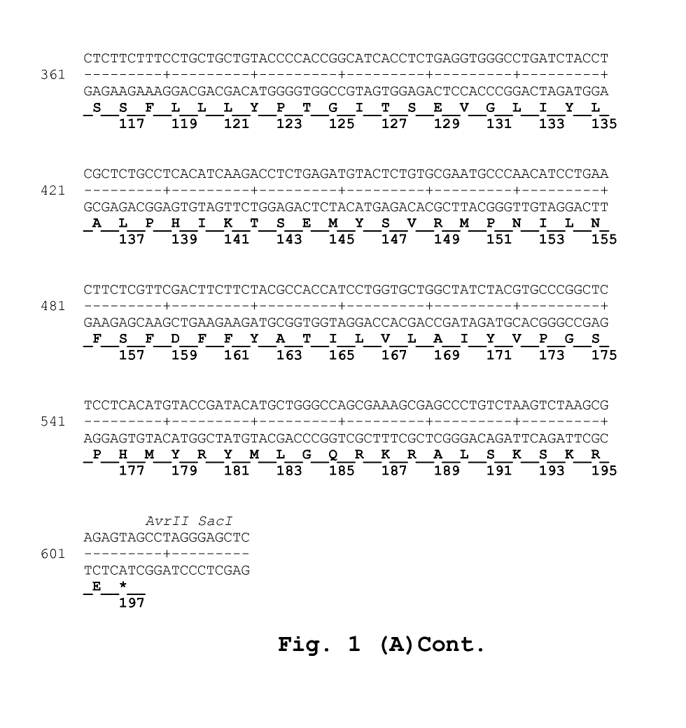

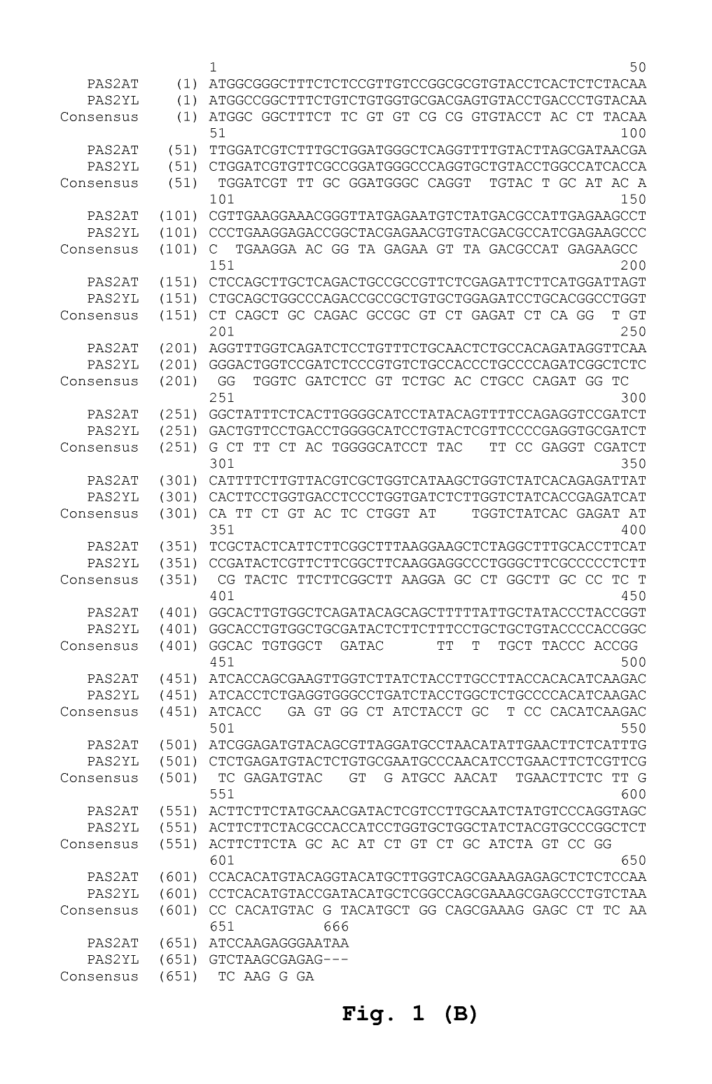

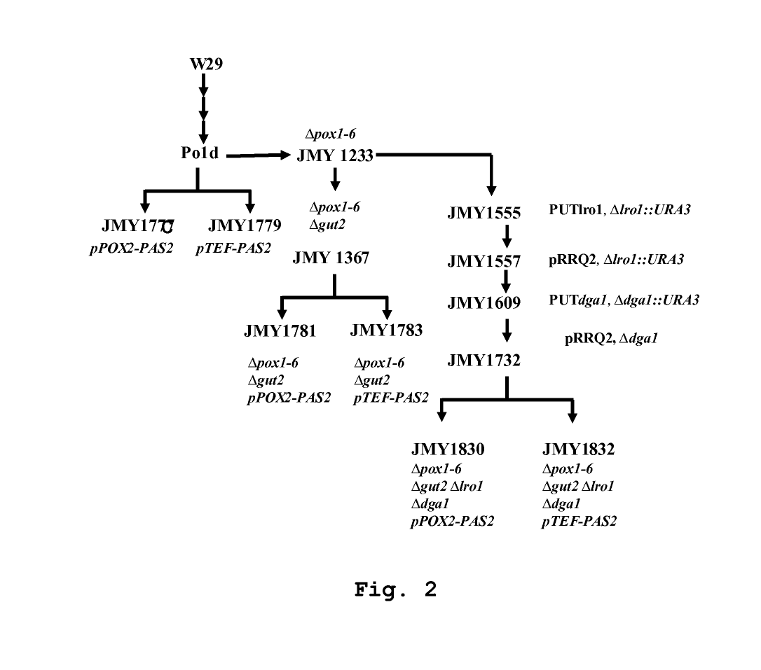

The open reading frame of Arabidopsis PAS2 gene was recoded to improve its expression with Yarrowia codon usage (FIG. 1A-B). Two restriction sites was added to facilitate cloning, BamHI and AvrII respectively at the 5′ and the 3′ end of PAS2 ORF. The new sequence, renamed PAS2^{Y1} was chemically synthetized (GeneArt inc.) and cloned into the two expression vectors JME1110 and JME1108. The two vectors allow the expression of PAS2 under a constitutive promoter (pTEF, JME1110) or oleic acid inducible promoter (pPOX2, JME1108).

Both constructs were used to transform the wild type strain Po1d (JMY195) and the Δgut2, Δpox1-6 obese strain (JMY1367). Transformants were selected on uracil and integration of the expression casette were verified by PCR. Several clones were selected and used for further analysis. However, since POX2 promoter allow strong expression even in absence of inducer, we mainly characterized transformants with JME1108 construct. The strains JMY1777 and JMY1778 are two independent clones of Po1d transformed with pPOX2-PAS2. Similarly, JMY1781 and JMY1782 are two independent clones of JMY1367 (Δgut Δpox1-6) transformed with pPOX2-PAS2.

### PAS2 Expression Improves Cell Growth and Leads to Lipid Body Fragmentation

The growth of different PAS2 expressing strains were compared with their untransformed relatives on glucose supplemented media. All the strains were inoculated at OD600=0.6 in 30 ml of YPD medium. All the strains showed a lag phase of about 4 to 5 hours and a bimodal curve with a plateau a plateau at 9 to 12 hours after inoculation before to reach the beginning of the stationary phase after 40 hours of culture.

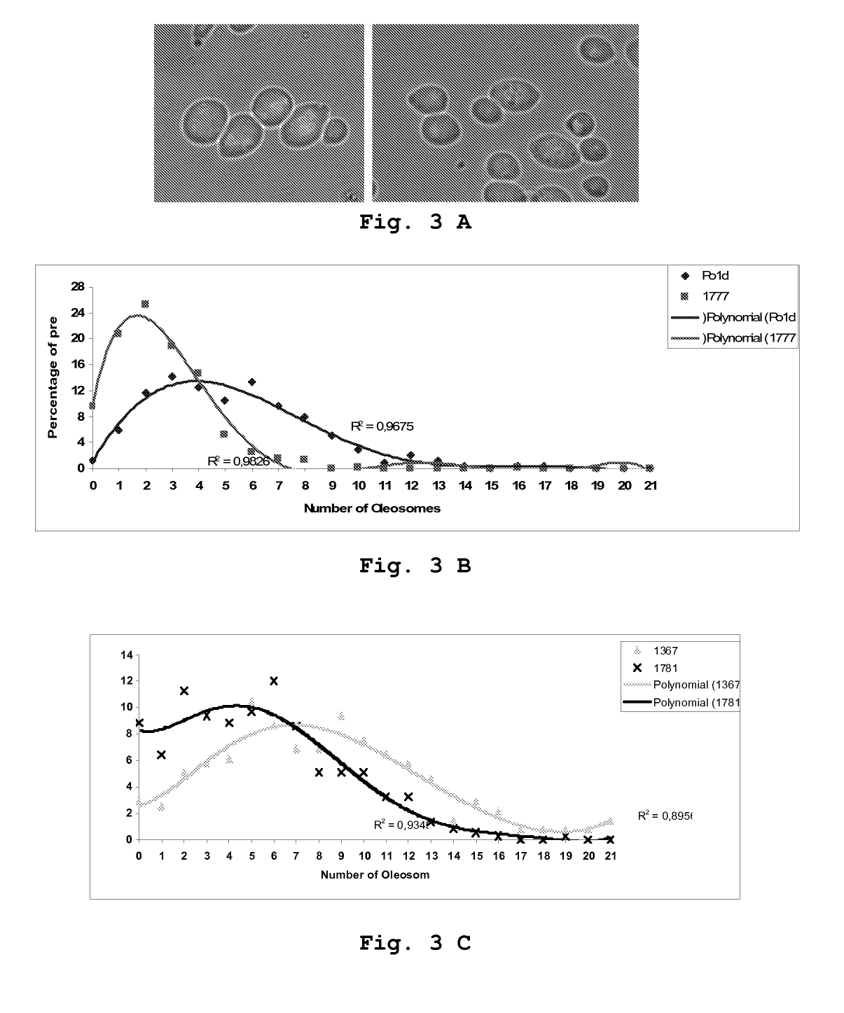

Yarrowia lipolytica is known to accumulate lipids in lipid bodies. It was reported that the obese strain JMY1367 was characterized by fusion of the lipid bodies in a larger structure. The effect of PAS2 expression on the structure of the lipid bodies was thereby checked by staining the different strains with Nile red (FIG. 3A). Cells were collected at 48 h since stationary stage is characterized with high accumulation of lipids, and the total number of lipid bodies per cell was quantified (FIG. 3B-C).

The expression of PAS2 in both Po1d as well as in the obese JM1367 leads to a reduction of number of lipid bodies (FIG. 3B-C).

### PAS2 Expression Enhances VLCFA Levels

Total lipid content was analysed by gas chromatography of fatty acyl methyl esters (FAMES) in the four strains at 3 different time point of growth curve, at the end of the first growth phase (11 h), at the end of second growth phase (24 h) and during stationary phase (48 h). As expected the obese strain JMY1367 has a higher fatty acid content compared to wild type Po1d with 22% and 40% at 24 h and 48 h respectively (FIG. 4). The expression of PAS2 reduced total fatty acid content at every time point. The strongest reduction was observed at 48 h with 20% in Po1d background and 46% in JMY1367 background.

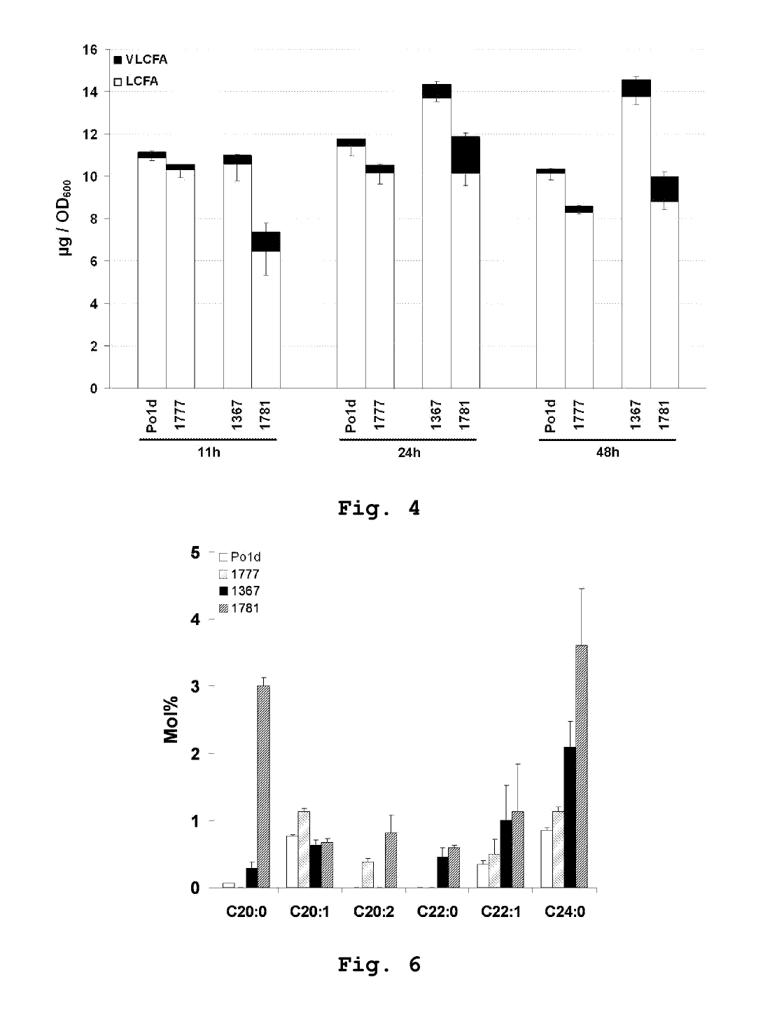

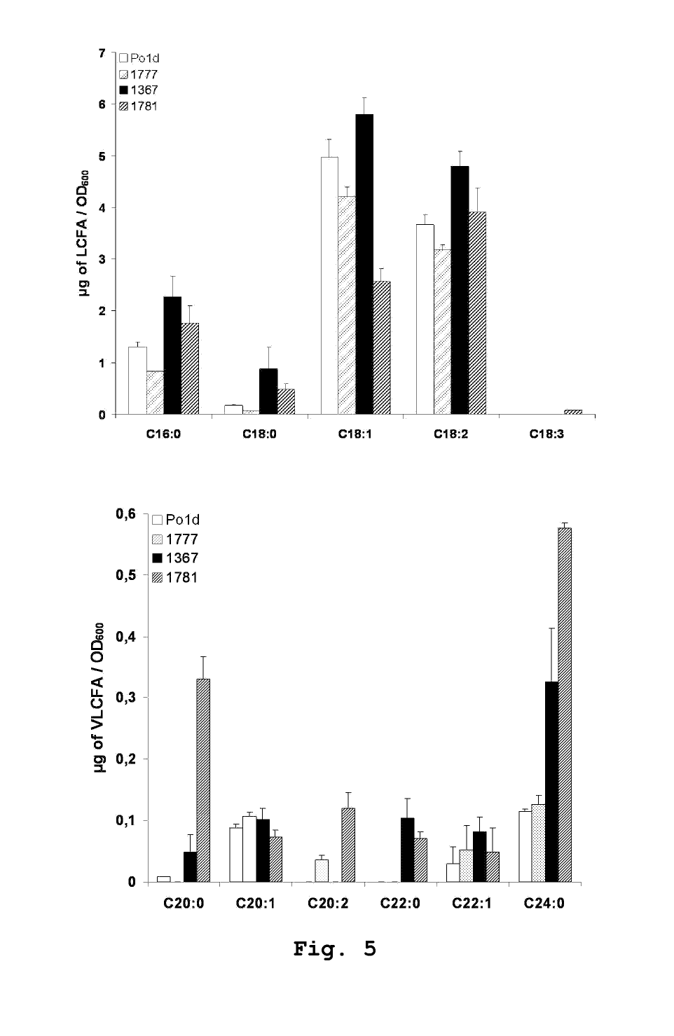

The amount of total long chain fatty acids (LCFA) which represent the most abundant fatty acids of Yarrowia lipolytica, was reduced by 18 and 36% in PAS2 expressing strains. Analysis of LCFA showed that all the different classes showed reduced levels upon PAS2 expression except that c18:1, which is one of the most abundant LCFA, was the most affected with for instance 126% reduction at 48 h in the obese JMY1367 background (FIG. 5). The reduction in total LCFA was effective even at the beginning of the growth curve (11 h).

VLCFA represent only minor lipid species in Yarrowia lipolytica (2.2-3.2% total fatty acids) (FIG. 4). Three major species were significantly accumulated: 24:0, 20:1 and 22:1 representing respectively 0.86, 0.77 and 0.34% of total fatty acids (Mol %) at 48 h of culture (FIG. 6). The Δgut Δpox1-6 had a clear effect on VLCFA levels since it doubled in 24 h of culture (6.53 μg/10OD compared to 3.23 in Po1d). The main VLCFA involved were c24:0 and c22:1 content that reached respectively 2.09 and 0.99% (Mol %) of total fatty acids. A new VLCFA could be detected as c22:0 reaching 0.45%. The expression of PAS2 in Po1d background did not change much the quantity or the nature of VLCFA accumulated. However, the expression of PAS2 in the obese JMY1367 background, increased very significantly VLCFA content. After 24 h of culture, JMY1781 accumulated 17.35 μg/10OD which was 2.65 and 5.3 fold more than the obese and wild type Po1d strains, respectively. The main VLCFA accumulated were c20:0 and c24:0 representing more than half of total VLCFA. Erucic acid c22:1, c22:0, c20:2 were also significantly accumulated in JMY1781.

### PAS2 Expression Induce the Accumulation of New monomethyl Branched Fatty Acids

Detail analysis of FAMES revealed that PAS2 expressing strain JMY1781 was accumulating new fatty acids. Mass spectrometry determined that lipids were monomethyl branched fatty acids with even or odd acyl chains. The JMY1781 showed in particular the presence of c14:0(Me), c15:0(Me), c16:0(Me), c17:0(Me), c18:0(Me) and c19:0(Me). The compounds were almost undectable in the wild type Po1d but also in the obese JMY1367 strains. The tetradecanoic acid, 12 methyl, methyl ester, 14:0(Me), appeared to be highly accumulated (at least to the level of Octadecanoic acid, methyl ester, c18:0). Several other products were accumulated in JMY1781 strain like the peaks at 10.7 min, 14.4 min, 18.4 min and 23 min.

### Discussion

The expression of the 3hydroxyacylCoA dehydratase PASTICCINO from Arabidopsis in Yarrowia lipolytica led to several innovative traits concerning the use of this yeast as a cell factory for oleo chemical biotechnology.

1—The expression of PAS2^{Y1} modifies oil body numbers in two different Yarrowia strains: a wild type Po1d but also the Δgut Δpox1-6 characterized by high accumulation of fatty acids inside the cell. Reduction of the lipid bodies number does not impair VLCFA accumulation. The reduction of lipid body number might improve oil extraction through press processing. Possibility, PAS2 might modify lipid secretion.

2—The expression of PAS2^{Y1} causes a very significant increase in VLCFA accumulation. Levels of VLCFA that could be used directly for industrial production should be obtained by co-expressing in Yarrowia sp. the other genes of the elongase complex such as known by the man skilled in the art. Since the expression of an Arabidopsis gene is efficient for changing VLCFA homeostasis in Yarrowia, we propose to use the other elongase genes from plants.

TABLE 1
Strains and plasmids used in this study
TABLE 1. Strains and plasmids used in this study

| | | |
|---|---|---|
| | | Reference or |
| Strain (host strain) | Plasmid, genotype | source |

E coli strains

| | | |
|---|---|---|
| DH5α | Φ80dlacZΔm15, recA1, endA1, gyrA96, thi-1, hsdR17 (rₖ−, mₖ+), | Promega |
| | supE44, relA1, deoR, Δ(lacZYA-argF)U169 | |
| JME461 (DH5α) | pRRQ2 (cre ARS68 LEU2 in pBluescript II KS+) | Fickers and al |
| | | 2003 |
| JME803 (DH5α) | JMP62-URA3ex, expression vector with the excisable URA3ex | Nicaud and al, |
| | marker and the POX2 promoter. | 2002 |
| JME1012 (DH5α) | JMP62-URA3ex, expression vector with the excisable URA3ex | This work |
| | marker and the TEF promoter. | |
| JME1107 (DH5α) | Synthetic PAS2 gene optimised with the codon usage of Y. lipolytica. | Geneart |
| JME1108 (DH5α) | PAS2-URA3, expression vector with the URA3ex marker under the | This work |
| | pPOX2 promoter inducible by oleic acid. | |
| JME1110(DH5α) | PAS2-URA3, expression vector with the URA3ex marker under a | This work |
| | constitutive promoter pTEF | |

Y. lipolytica strains

| | | |
|---|---|---|
| JMY399, W29 | MATA, wild-type | Barth and |
| | | Gaillardin, |
| | | 1996 |
| JMY195, Po1d | MATA ura3-302 leu2-270 xpr2-322 | Barth and |
| | | Gaillardin, |
| | | 1996 |
| MTLY95a, JMY1233 | MATA ura3-302 xpr2-322 Δleu2 Δpox1-6 | Thevenieau et |
| | | al, 2004 |
| JMY1367 | MATA ura3-302 xpr2-322, Δleu2 Δpox1-6 Δgut2 | Beopoulos, |
| | | 2008 |
| JMY1732 | MATA ura3-302 xpr2-322 Δleu2 Δpox1-6 Δlro1 Δdga1 | This work |
| JMY 1777 | Po1d, JMP62-URA3ex-pPOX2-PAS2 | This work |
| JMY 1779 | Po1d, JMP62-URA3ex-pTEF-PAS2 | This work |
| JMY 1781 | JMY1367, JMP62-URA3ex-pPOX2-PAS2 | This work |
| JMY 1783 | JMY1367, JMP62-URA3ex-pTEF-PAS2 | This work |
| JMY 1830 | JMY 1732, JMP62-URA3ex-pPOX2-PAS2 | This work |
| JMY 1832 | JMY 1732, JMP62-URA3ex-pTEF-PAS2 | This work |

TABLE II
Primers used in this study

| | | Restriction |
|---|---|---|
| | | site, |
| Primers | Sequence (5′ → 3′)^{a} | introduced |
| LRO1-ver1 | CCACGGAGACTGGAACAGATGTCGG | |
| SEQ ID N^{o} 1 | | |
| LRO1-P1 | GGATCCCAGTGCTCTAGACTGTC | |
| SEQ ID N^{o} 2 | | |
| LRO1-P2 | GCTAGGGATAACAGGGTAATGCGCGGTAGCTGAGACATGTCGCGTG | IsceI |
| SEQ ID N^{o} 3 | | |
| LRO1-T1 | GCATTACCCTGTTATCCCTAGCGCGTTCGTCCTCTCATGATTCC | IsceI |
| SEQ ID N^{o} 4 | | |
| LRO1-T2 | CCAAACATAGTCATTTGCGGATCC | |
| SEQ ID N^{o} 5 | | |
| LRO1-ver2 | CCAAGGGACCGTCTGGCCAAAC | |
| SEQ ID N^{o} 6 | | |
| DGA1-ver1 | CGGACACCTCTTTTATGCTGCGGGC | |
| SEQ ID N^{o} 7 | | |
| DGA1-P1 | GGCGGATCCTGGTGCATTTTTGC | |
| SEQ ID N^{o} 8 | | |
| DGA1-T1 | GCTAGGGATAACAGGGTAATGCGCAAACTCATCTGGGGGAGATCC | IsceI |
| SEQ ID N^{o} 9 | | |
| DGA1-P2 | GCATTACCCTGTTATCCCTAGCGAGCTTATCAGTCACGGTCCACG | IsceI |
| SEQ ID N^{o} 10 | | |
| DGA1-T2 | CCATAGAGGTGTCCCCAAACG | |
| SEQ ID N^{o} 11 | | |
| DGA1-ver2 | CCCCCAAGCATACCGACCGTCGC | |
| SEQ ID N^{o} 12 | | |
| 61start^{b} | CTTATATACCAAAGGGATGGGTC | |
| SEQ ID N^{o} 13 | | |
| 61stop^{b} | GTAGATAGTTGAGGTAGAAGTTG | |
| SEQ ID N^{o} 14 | | |
| TEFstart^{b} | GGGTATAAAAGACCACCGTCC | |
| SEQ ID N^{o} 15 | | |

^{a}underlined sequences correspond to introduced restriction sites

### REFERENCES

Bach L, Michaelson L V, Haslam R, Bellec Y, Gissot L, Marion J, Da Costa M, Boutin J P, Miguel M, Tellier F, Domergue F, Markham J E, Beaudoin F, Napier J A, Faure J D. (2008): “The very-long-chain hydroxy fatty acyl-CoA dehydratase PASTICCINO2 is essential and limiting for plant development. >> Proc Natl Acad Sci U S A. 2008 Sep 23;105(38):14727-31

Barth and Gaillardin.: Yarrowia lipolytica, in: Nonconventional Yeasts in Biotechnology A Handbook (Wolf, K., Ed.), Vol. 1, 1996, pp. 313-388. Springer-Verlag).

Beopoulos, A., Mrozova, Z., Thevenieau, F., Le Dall, M. T., Hapala, I., Papanikolaou, S., Chardot, T., and Nicaud, J. M. (2008). Control of lipid accumulation in the yeast Yarrowia lipolytica. Appl Environ Microbiol 74, 7779-7789.

Beopoulos, A., Cescut, J., Haddouche, R., Uribelarrea, J. L., Molina-Jouve, C., and Nicaud, J. M. (2009). Yarrowia lipolytica as a model for bio-oil production. Prog Lipid Res 48, 375-387.

Blanchin-Roland et al., Two Upstream Activation Sequences Control the Expression of the XPR2 Gene in the Yeast Yarrowia lipolytica, Molecular and Cellular Biology, vol. 14 (1):327-338, 1994.

Denic V, Weissman J S. (2007): “A molecular caliper mechanism for determining very long-chain fatty acid length.” Cell. 2007 Aug 24;130 (4):663-77.

Fickers P., Le Dall M. T., Gaillardin C. Thonart P. Nicaud J-M. (2003) New disruption cassettes for rapid gene disruption and marker rescue in the yeast Yarrowia lipolytica. J. Microbiol. Methods 55/3:727-737.

Juretzek T, Wang H., Nicaud, J-M., Mauersberger S, Barth G. (2000). Comparison of promoters suitable for regulated overexpression of β-galactosidase in the alkane-utilizing yeast Yarrowia lipolytica. Biotechnol. Bioprocess Eng. 5:320-326.

Leblond, Y., A. Marty, N. Mouz, and J. L. Uribelarrea. 2009. Method for producing lipase, transformed Yarrowia lipolytica cell capable of producing said lipase and their uses.

Madzak, C., Gaillardin, C. and Beckerich, J. M. (2004). J. Biotechnol. 109:63-81. (review).

Madzak, C., Treton, B. and Blanchin-Roland, S. (2000). J. Mol. Microbiol. Biotechnol. 2:207-216.

Mauersberger, S., Nicaud, J-M (2002) Chapter 56. Tagging of genes by insertional mutagenesis in the yeast Yarrowia lipolytica. In: Laboratory Manual on Non-conventional Yeasts in Genetics, Biochemistry and Biotechnology.

Nicaud J-M., Fabre E., Gaillardin C. (1989) Expression of invertase activity in Yarrowia lipolytica and its use as a selective marker. Cur. Genet. 16:253-260.

Nicaud, J-M, Madzak, C., van den Broek, P., Gysler, C., Duboc, P., Niederberger, P. Gaillardin, C. (2002) Protein expression and secretion in the yeast Yarrowia lipolytica. FEMS Yeast Research 2/3:371-379.

Wolf K, Breunig K, Barth G (eds), Springer, Berlin Heidelberg-New York, pp 343-356. (Buchbeitrag).

1. A recombinant strain of a Yarrowia sp., which comprises a heterologous gene coding for a hydroxyacyl-CoA dehydratase, under control of regulatory elements allowing expression of the said heterologous gene in the Yarrowia sp.

2. The recombinant strain of claim 1, wherein the gene coding for the hydroxyacyl-CoA dehydratase is selected from the group consisting of genes of plant species coding for a hydroxyacyl-CoA dehydratase, and functional homologues and fragments thereof.

3. The recombinant strain of claim 2, wherein the gene of plant species is selected from the group consisting of the genes coding for a hydroxyacyl-CoA dehydratase from Arabidopsis thaliana, Vitis vinifera, Oryza sativa, Brassica rapa, Hyacinthus orientalis, Ostreacoccus lucimarinus, Chlamydomonas reinhardtii, Brassica napus, Raphanus sativus, and Brassica oleracea.

4. The recombinant strain of claim 1, wherein the heterologous gene coding for a hydroxyacyl-CoA dehydratase is the gene PAS2 from Arabidopsis thaliana.

5. The recombinant strain of claim 1, wherein the strain of Yarrowia sp. belongs to the genus Yarrowia lipolytica.

6. The recombinant strain of claim 1, wherein the recombinant strain further comprises deletion of at least one gene involved in the β-oxidation of fatty acids.

7. The recombinant strain of claim 6, wherein the deletion of the at least one gene involved in the β-oxidation of fatty acids is deletion of a gene coding for a glucose 3-phosphate dehydrogenase, and/or the gene POX1-6 or both.

8. A method for the production of Very Long Chain Fatty Acids (VLCFA) by fermentation, comprising culturing a recombinant strain of claim 1 in an appropriate culture medium and recovering the VCLA from the strain, the medium, or both.

9. (canceled)

10. The method of claim 8, wherein the heterologous gene coding for a hydroxyacyl-CoA dehydratase is a gene selected from the group consisting of the genes coding for a hydroxyacyl-CoA dehydratase from Arabidopsis thaliana, Vitis vinifera, Oryza sativa, Brassica rapa, Hyacinthus orientalis, Ostreacoccus lucimarinus, Chlamydomonas reinhardtii, Brassica napus, Raphanus sativus, and Brassica oleracea.

11. The method of claim 8, wherein the heterologous gene coding for a hydroxyacyl-CoA dehydratase is the gene PAS2 from Arabidopsis thaliana.

12. The method of claim 8, wherein the recombinant strain further comprises deletion of at least one gene involved in the β-oxidation of fatty acids.

13. The method of claim 12, wherein the deletion of the at least one gene involved in the β-oxidation of fatty acids is deletion of a gene coding for a glucose 3-phosphate dehydrogenase, and/or the gene POX1-6, or both.

14. The method of claim 8 wherein the recombinant strain of Yarrowia sp. belongs to the genus Yarrowia lipolytica.

15. A VLCFA composition obtained by the method of claim 8.

16. The VLCFA composition of claim 15 which comprises monomethyl branched fatty acids with even or odd acyl chains.
